(19) United States
(12) Patent Application Publication
(10) Pub. No.: US 2015/0202385 A1
(43) Pub. Date: Jul. 23, 2015

(54) FLUORESCENCE-BASED OPTICAL SENSOR FOR DETECTING INFUSION PUMP CASSETTE

(71) Applicant: Zevex, Inc., Salt Lake City, UT (US)

(72) Inventors: Jeffery T. Juretich, Herriman, UT (US); Michael Marshall, Herriman, UT (US); Michael Elwood, Farmington, UT (US); Daniel A. Martel, Stansbury, UT (US); Jeffrey Geisler, Salt Lake City, UT (US)

(21) Appl. No.: 14/161,845

(22) Filed: Jan. 23, 2014

(51) Int. Cl. A61M 5/50; A61M 5/142

(52) U.S. Cl. CPC A61M 5/5086; A61M 5/142

(57) ABSTRACT

An infusion pump has an optical cassette detection system for determining whether or not a cassette of an administration tubing set is properly loaded in the pump. Operation of the pump may be enabled or disabled based on a determination of the cassette detection system. The cassette detection system includes a light emitter and a corresponding photosensitive detector, and a window carried by the cassette that includes a fluorophore. When the cassette is properly loaded in the pump, an excitation light beam from the emitter enters the window and excites the fluorophore, causing emission light in a wavelength band distinct from that of the excitation beam to be transmitted out of the window for receipt by the detector. The detector signal is evaluated by signal evaluation electronics to determine if the signal level is above a predetermined threshold, indicating the cassette is loaded.

### FIELD OF THE INVENTION

The present invention relates generally to infusion pumps for controlled delivery of liquid food and medications to patients. More specifically, the present invention relates to a sensor system in an infusion pump for detecting the presence or absence of a cassette by which an administration tubing set is operatively connected to the pump.

### BACKGROUND OF THE INVENTION

Programmable infusion pumps are used to carry out controlled delivery of liquid food for enteral feeding and medications for various purposes, for example pain management. In a common arrangement, an infusion pump receives a disposable administration set comprising a cassette removably received by the pump and flexible tubing connected to the cassette for providing a fluid delivery path through the pump.

The cassette itself may be intended for use with a particular infusion pump model or models, and/or with tubing having predetermined properties. In this regard, the cassette may include safety features that are designed and manufactured according to specifications determined at least in part by the intended infusion pump model and/or administration set tubing. The safety features of the cassette may cooperate with corresponding features on the matching pump, and may be manufactured according to size tolerances related to tubing diameter and flexibility. For example, the cassette may have an anti-free flow mechanism for protecting the patient from uncontrolled fluid delivery. The anti-free flow mechanism may take the form of an external pinch clip occluder actuated when the cassette is properly loaded in the pump and a door of the pump is closed. Alternatively, the anti-free flow mechanism may take the form of an internal “in-line occluder” that resides within the flow passage of the tubing, wherein a flow passage is only opened when the cassette is properly loaded in the pump and the pump door is closed.

The cassette may provide additional safety features beyond free flow protection. For example, the cassette may be matched to the pump to maintain a desired volumetric accuracy of the pump, and to ensure correct function of occlusion and air-in-line sensors used to trigger safety alarms.

In view of the safety importance of the cassette, it is desirable to provide means to detect whether or not a matching cassette is properly loaded in the pump as a precondition to enabling pump operation.

### SUMMARY OF THE INVENTION

In accordance with the present invention, an infusion pump in which an administration set is removably received is provided with an optical detection system for determining whether or not a cassette of the administration set is properly loaded in the pump. In an embodiment of the present invention, operation of the pump is disabled if a cassette is not properly loaded in the pump.

The optical cassette detection system generally comprises an optical emitter and a corresponding photosensitive detector each mounted to the pump, and a window carried by the cassette that includes at least one fluorophore. The optical emitter is arranged to emit an excitation light beam directed along an optical axis, wherein the excitation light beam is in an excitation wavelength band chosen to excite the at least one fluorophore in the window. The window intersects the optical axis at a location between the optical emitter and the photosensitive detector when the cassette is properly loaded in the pump so that the at least one fluorophore is exposed to the excitation light beam and emits light in an emission wavelength band distinct from the excitation wavelength band. The photosensitive detector is arranged along the optical axis to receive light in the emission wavelength band exiting the window. The photosensitive detector is configured to detect light within the emission wavelength band, and generates a detector signal representing an intensity of light in the emission wavelength band which it receives.

The detector signal is evaluated by signal evaluation electronics to determine if the detector signal level is above a predetermined threshold, indicating that a cassette is properly loaded in the pump. The signal evaluation electronics may be in communication with a pump controller, wherein the pump controller is programmed to disable pump operation unless a cassette is loaded as determined by the optical cassette detection system.

In an embodiment of the invention, the window includes a light entry surface and a light exit surface parallel to the light entry surface, and the window is integrally formed with the cassette in a one-piece molded part made of transparent plastic or translucent plastic that is doped with one or more fluorophores.

### BRIEF DESCRIPTION OF THE DRAWINGS

The nature and mode of operation of the present invention will now be more fully described in the following detailed description of the invention taken with the accompanying drawing figures, in which:

FIG. 1 is perspective view of an infusion pump and cassette incorporating a cassette detection system in accordance with an embodiment of the present invention;

FIG. 2 is a perspective view of the cassette shown in FIG. 1;

FIG. 3A is a schematic sectional view illustrating a cassette detection system formed in accordance with an embodiment of the present invention, wherein a tab of the cassette is shown prior to insertion into a tab-receiving slot of the pump;

FIG. 3B is an enlarged view corresponding to FIG. 3A, however the cassette tab is shown inserted into the pump slot;

FIG. 4 is a flow diagram showing decision logic executed by the cassette detection system in accordance with an embodiment of the present invention.

### DETAILED DESCRIPTION OF THE INVENTION

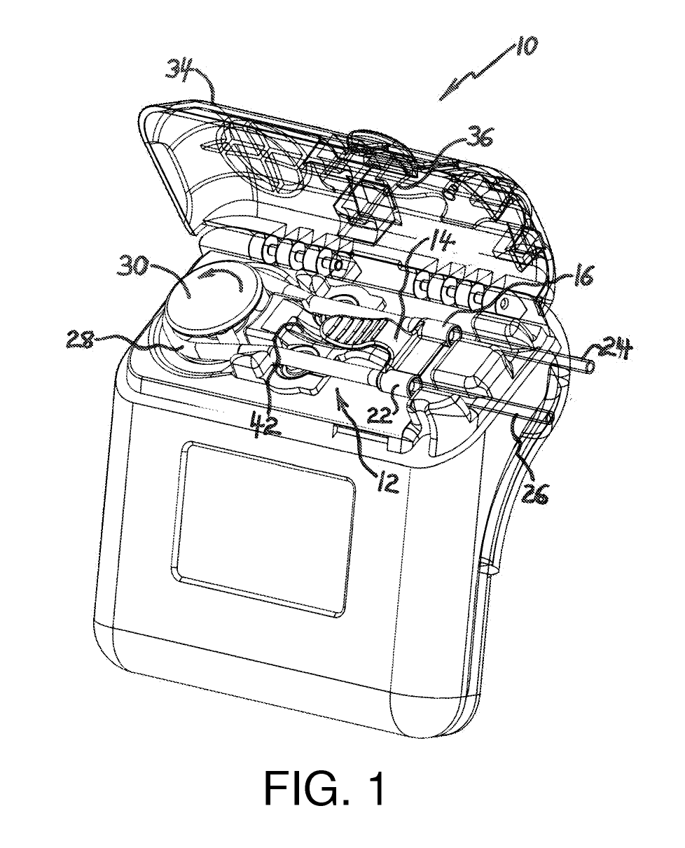

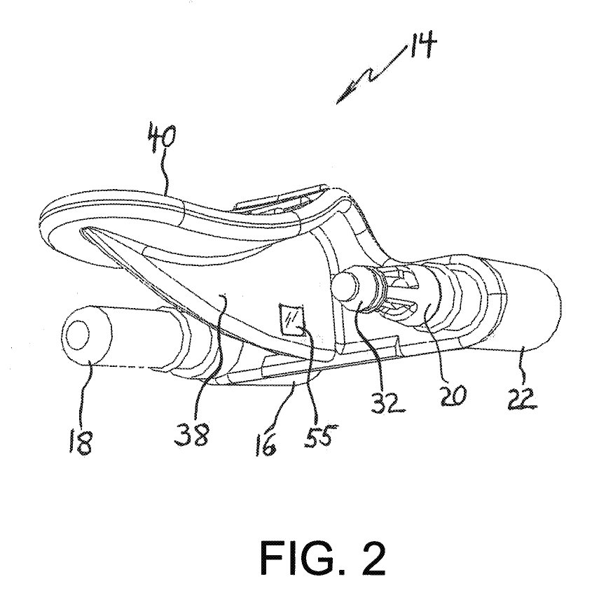

FIG. 1 shows an infusion pump 10 in which an administration set 12 is removably received. Administration set 12 includes a cassette 14, which is shown by itself in FIG. 2. Cassette 14 may include an input connector 16, an upstream loop connector 18 in flow communication with input connector 16, a downstream loop connector 20, and an output connector 22 in flow communication with downstream loop connector 20. Administration set 12 may further include inflow tubing 24 having one end mated to input connector 16 and an opposite end (not shown) connected to a fluid source, and outflow tubing 26 having one end connected to output connector 22 and an opposite end (not shown) connected to a patient. Finally, administration set 14 may further include a pumping segment of tubing 28 having one end mated to upstream loop connector 18 and an opposite end mated to downstream loop connector 20.

In the illustrated embodiment, pump 10 is a rotary peristaltic pump having a rotor 30, wherein pumping segment 28 is wrapped around rotor 30 and is engaged by angularly spaced rollers on rotor 30 as the rotor rotates to provide peristaltic pumping action forcing liquid through the tubing of administration set 12. As may be understood by reference to FIG. 1, when rotor 30 rotates in a counter-clockwise direction, liquid is moved from inflow tubing 24 through input connector 16 and upstream loop connector 18 to pumping segment 28, and then from pumping segment 28 through downstream loop connector 20 and output connector 22 to outflow tubing 26. Although the present invention is described in the context of a rotary peristaltic pump, the invention is not limited to this type of infusion pump. The invention may be practiced with any type of infusion pump that receives an administration set having a cassette.

Cassette 14 may include an in-line occluder 32 which may be incorporated into downstream loop connector 20. In-line occluder 32 prevents flow when pump door 34 is open. An actuator 36 on an underside of pump door 34 engages pumping segment 28 in a manner which opens a flow path around occluder 32 when door 34 is closed.

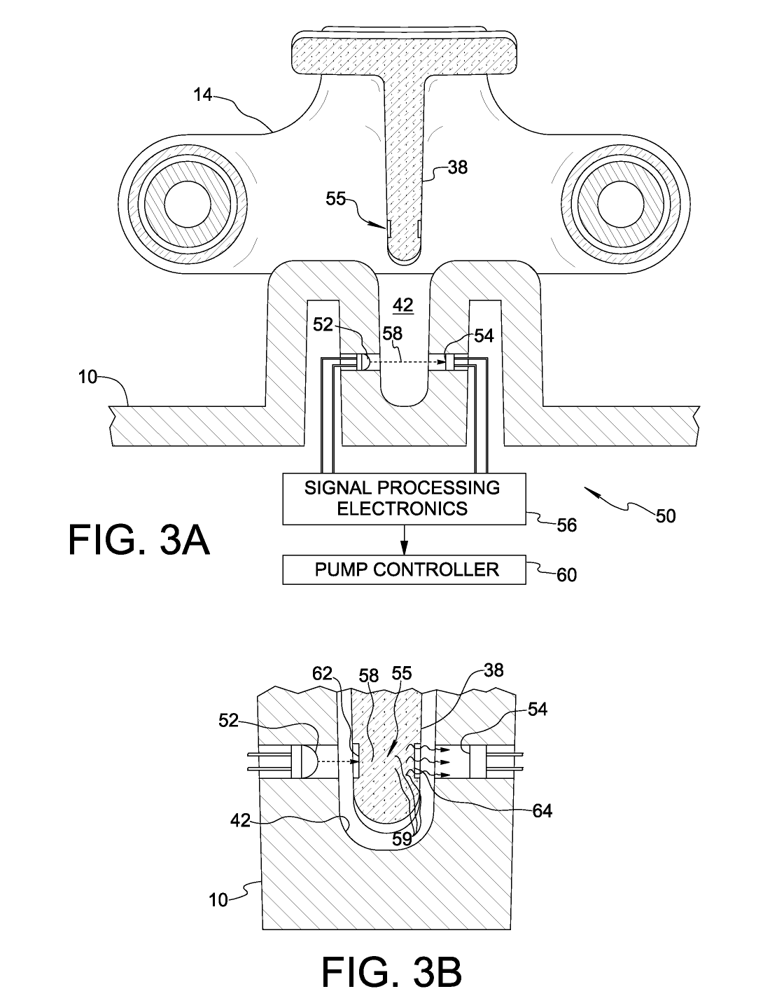

Reference is now made to FIGS. 3A and 3B. Cassette 14 includes a tab 38 depending downwardly from a ribbed thumb portion 40 of the cassette. In the present embodiment, tab 38 is a generally planar tab that is sized for receipt within a corresponding slot 42 in pump 10. Slot 42 may be provided at a location on pump 10 between the upstream and downstream portions of pumping segment 28, and tab 38 may be provided on an underside of thumb portion 40. For example, slot 42 may be midway between the upstream and downstream portions of pumping segment 28 and may be elongated in a direction aligned with the rotation axis of rotor 30, and tab 38 may be midway between one side of cassette 14 having input connector 16 and upstream loop connector 18 and the other side of cassette 14 having downstream loop connector 20 and output connector 22. In this symmetrical arrangement, cassette 14 is easily centered in pump 10 relative to rotor 30 during installation of administration set 12. In an embodiment of the invention, the width of slot 42 is 2.6 mm and the width of tab 38 is 1.7 mm.

Pump 10 includes an optical cassette detection system 50 operable to detect whether or not cassette 14 is properly loaded in pump 10 with cassette tab 38 present in slot 42. Cassette detection system 50 includes an optical emitter 52, which may be mounted to pump 10 on one side of slot 42, and a photosensitive detector 54, which may be mounted to pump 10 on an opposite side of slot 42. In the illustrated embodiment, detector 54 is aligned with emitter 52 along an optical axis 58 passing through slot 42, however detector 54 may be arranged so that it is not aligned with emitter 52 along optical axis 58. Cassette detection system 50 further includes a window 55 carried by cassette 14. Window 55 is arranged on cassette 14 to intersect optical axis 58 at a location between optical emitter 52 and photosensitive detector 54 when cassette 14 is properly loaded in pump 10. Cassette detection system 50 may also include signal processing electronics 56 connected to photosensitive detector 54 for receiving an electronic signal generated by detector 54 and evaluating the signal. Signal processing electronics 56 may be in communication with a pump controller 60, whereby operation of pump 10 may be controlled based on an evaluation of the detector signal.

In accordance with the present invention, window 55 includes at least one fluorophore 59, and optical emitter 52 is arranged to emit an excitation light beam directed along optical axis 58. The excitation light beam is in an excitation wavelength band chosen to excite the fluorophore(s) 59, whereby the fluorophore(s) emit light in an emission wavelength band distinct from the excitation wavelength band in response to absorption of excitation beam energy. In the present specification, reference to the emission wavelength band being “distinct from” the excitation wavelength band means that the emission wavelength band and the excitation wavelength band are respectively centered about two different wavelengths that are distinguishable from one another. Emitter 52 may be a light-emitting diode (LED) or other light source. Emitter 52 may be a narrow band emitter, for example a laser LED, emitting light in the excitation wavelength band. Alternatively, emitter 52 may emit light in a relatively wide wavelength band, and a wavelength filter (no shown) may be arranged after the emitter to filter the light by passing only light in the excitation wavelength band.

Photosensitive detector 54 may be configured to detect light within the emission wavelength band. For example, detector 54 may have a spectral responsivity that is substantially confined to the emission wavelength band or some spectral portion thereof, and that drops off significantly for the excitation wavelength band. Alternatively, or in addition, detector 54 may be configured with a wavelength filter (not shown) that filters out light in the excitation wavelength band and passes light in the emission wavelength band or in some portion of the emission wavelength band for detection. Photosensitive detector 54 generates a detector signal, for example a current or voltage signal, having a level corresponding to the intensity of light in the emission wavelength band received by the detector. Photosensitive detector 54 may be a photodiode or other photosensitive element capable of generating an electrical signal in response to incident light.

In the embodiment shown herein, emitter 52 and photosensitive detector 54 are each mounted in pump 10 adjacent to slot 42, and window 55 is part of tab 38, however other configurations and arrangements are possible. While not shown, emitter 52 and detector 54 may have lenses, fiber optics, or other optical elements associated therewith for collimating, focusing, and/or directing the beam.

As best seen in FIG. 3B, window 55 may include a light entry surface 62 normal to optical axis 58, and a light exit surface 64 also normal to optical axis 58. Window 55 may be integrally formed with tab 38 or with cassette 14 as a whole, wherein surfaces 62 and 64 are formed as external surface features of the molded part. For example, cassette 14 may be molded from transparent or translucent optical grade plastic that is doped with the fluorophore(s) 59. Possible fluorophores include, but are not limited to, phosphorescent materials. For example, YAG:Ce yellow phosphor may be used as a fluorophore, and has an excitation wavelength band centered at 450 nm and an emission wavelength band centered at 577 nm.

When cassette 14 is not loaded in pump 10, the excitation light beam from emitter 52 passes directly to detector 54. Because detector 54 is configured to detect light in the emission wavelength band, and not light in the excitation wavelength band, the level of the signal generated by detector 54 will remain below a predetermined threshold level when cassette 14 is not loaded. When cassette 14 is properly loaded in pump 10, the at least one fluorophore 59 in window 55 absorbs light in the excitation wavelength band and emits light in the emission wavelength band. Some of the light in the emission wavelength band is received by detector 54. Consequently, when cassette 14 is loaded in pump 10, the level of the signal generated by detector 54 will rise above the threshold level.

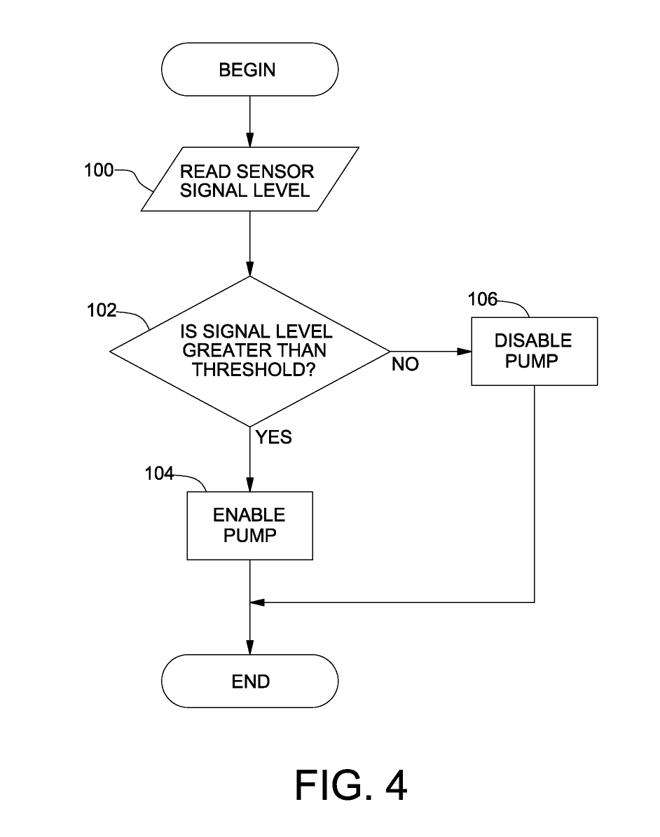

Signal processing electronics 56 evaluates the signal from detector 54 to determine if cassette 14 is properly loaded in pump 10. The signal processing and evaluation may be completely analog, or the detector signal level may be converted to a digital value and compared to a threshold in a digital comparator circuit. As illustrated in FIG. 4, operation of pump 10 may be enabled or disabled based on the determination made by signal processing electronics 56. In block 100, the level of the detector signal is read. In block 102, the signal level is compared to a predetermined threshold as the basis for a decision. If the signal level is above the threshold, loading of cassette 14 is indicated and flow branches to block 104, wherein pump operation is enabled by pump controller 60. However, if the signal level is below the threshold, flow branches to block 106 and pump operation is disabled by pump controller 60.

Tab 38 on cassette 14 provides structure that may be used for carrying window 55 and positioning the window in optical cassette detection system 50. A wide variety of tab arrangements and optical detection system configurations are of course possible. The centered arrangement of a thin tab 38 on the underside of cassette 14, and the use of a thin slot 42 in pump 10, takes advantage of the tab and slot as a means for guiding and centering the cassette 14 during installation. Moreover, the cassette detection system 50 is hidden within the pump and is inconspicuous to users. Emitter 52 and detector 54 may be recessed slightly from the surface of slot 42 behind respective transparent barriers (not shown) to keep dirt and fluid away from the emitter and detector.

While the invention has been described in connection with exemplary embodiments, the detailed description is not intended to limit the scope of the invention to the particular forms set forth. The invention is intended to cover such alternatives, modifications and equivalents of the described embodiment as may be included within the spirit and scope of the invention.

What is claimed is:

1. A system for detecting loading of a cassette in an infusion pump, the system comprising:
an optical emitter mounted to the pump, the optical emitter being arranged to emit an excitation light beam directed along an optical axis, wherein the excitation light beam is in an excitation wavelength band;
a photosensitive detector mounted to the pump, wherein the photosensitive detector is configured to detect light within an emission wavelength band distinct from the excitation wavelength band, wherein the photosensitive detector generates a detector signal representing an intensity of light in the emission wavelength band received thereby; and
a window carried by the cassette, the window intersecting the optical axis at a location between the optical emitter and the photosensitive detector when the cassette is properly loaded in the pump, wherein the window includes at least one fluorophore excitable by the excitation light beam, wherein the at least one fluorophore emits light in the emission wavelength band in response to excitation by the excitation light beam, wherein light in the emission wavelength band is received by the photosensitive detector.

2. The system according to claim 1, further comprising signal processing electronics for evaluating the detector signal to determine whether the cassette is properly loaded in the pump.

3. The system according to claim 2, wherein the window includes a light entry surface facing the optical emitter and a light exit surface facing the photosensitive detector, wherein the light entry surface and the light exit surface are normal to the optical axis.

4. The system according to claim 1, wherein the window is integrally formed with the cassette in a one-piece molded part.

5. The system according to claim 4, wherein the one-piece molded part is molded from transparent plastic or translucent plastic, and the plastic is doped with the at least one fluorophore.

6. The system according to claim 1, wherein the cassette includes a tab in which the window is located, and the pump includes a slot configured to receive the tab when the cassette is properly loaded in the pump.

7. The system according to claim 6, wherein the optical emitter and the photosensitive detector are on opposite sides of the slot.

8. The system according to claim 7, wherein the excitation wavelength band is centered about a wavelength of 450 nm and the emission wavelength band is centered about a wavelength of 577 nm.

9. The system according to claim 1, wherein the photosensitive detector is arranged along the optical axis.

10. An infusion pump comprising:
an optical emitter mounted to the pump, the optical emitter being arranged to emit an excitation light beam directed along an optical axis, wherein the excitation light beam is in an excitation wavelength band;
a photosensitive detector mounted to the pump, wherein the photosensitive detector is configured to detect light within an emission wavelength band distinct from the excitation wavelength band, wherein the photosensitive detector generates a detector signal representing an intensity of light in the emission wavelength band received thereby; and
wherein proper loading of a cassette in the infusion pump causes absorption of light in the excitation wavelength band and corresponding emission of light in the emission wavelength band for receipt by the photosensitive detector.

11. The infusion pump according to claim 10, further comprising signal processing electronics for evaluating the detector signal to determine whether a cassette is properly loaded in the pump.

12. The infusion pump according to claim 11, wherein the signal processing electronics is programmed to determine that a cassette is properly loaded in the pump when the level of the detector signal is above a predetermined threshold.

13. The infusion pump according to claim 10, wherein the photosensitive detector is arranged along the optical axis.

14. A cassette to be loaded in an infusion pump for operatively connecting an administration set to the pump, the cassette comprising:
an input tubing connector;
an output tubing connector;
a window spaced from the input tubing connector and the output tubing connector, wherein the window includes at least one fluorophore excitable by an excitation light beam in an excitation wavelength band.

15. The cassette according to claim 14, wherein the window includes a light entry surface and a light exit surface parallel to the light entry surface.

16. The cassette according to claim 14, wherein the window is integrally formed with the cassette in a one-piece molded part.

17. The cassette according to claim 16, wherein the one-piece molded part is molded from transparent plastic or translucent plastic, and the plastic is doped with the at least one fluorophore.
